(19) United States
(12) Patent Application Publication
(10) Pub. No.: US 2010/0136646 A1
(43) Pub. Date: Jun. 3, 2010

(54) Selective Inactivation Of Microorganisms With A Femtosecond Laser

(76) Inventors: Kong-Thon Tsen, Chandler, AZ (US); Shaw-Wei D. Tsen, Chandler, AZ (US); Juliann G. Kiang, Potomac, MD (US)

Correspondence Address: Ashok Tankha, 36 Greenleigh Drive, Sewell, NJ, 08080, US

(21) Appl. No.: 12/646,961

(22) Filed: Dec. 23, 2009

Related U.S. Application Data
(63) Continuation-in-part of application No. 12/131,710, filed on Jun. 2, 2008.
(60) Provisional application No. 60/932,668, filed on Jun. 1, 2007.

(51) Int. Cl. C12N 13/00

(52) U.S. Cl. 435/173.1

(57) ABSTRACT

A method is provided for selectively inactivating microorganisms with femtosecond pulsed lasers. Under proper laser conditions, irradiation of the femtosecond pulsed laser causes inactivation of pathogenic microorganisms, for example, viruses, bacteria and protozoa, without causing cytotoxicity in mammalian cells. Pathogenic microorganism activity is diminished through an impulsive stimulated Raman scattering process, that is, through the excitation of the low-energy vibrational state on the outer structure of a microorganism with femtosecond pulsed lasers. The wavelength of the laser pulses is in a range of the electromagnetic spectrum, for example, visible and near-infrared where water is substantially transparent. The method is utilized for cleansing blood components, disinfecting drinking water, treating viral and bacterial diseases, extracting nucleic acid from microorganisms, and for manufacturing vaccines.

### CROSS REFERENCE TO RELATED APPLICATIONS

This is a continuation in part application of non-provisional patent application Ser. No. 12/131,710, titled “System And Method For Inactivating Microorganisms With A Femtosecond Laser” filed on Jun. 2, 2008 in the United States Patent and Trademark Office, which claims the benefit of provisional patent application No. 60/932,668, titled “System and Method for diminishing the activity of microorganisms with a visible femtosecond laser” filed on Jun. 1, 2007 in the United States Patent and Trademark Office.

The specifications of the above referenced patent applications are incorporated herein by reference in their entirety.

### STATEMENT REGARDING FEDERALLY SPONSORED RESEARCH

The United States government has a paid-up license in this invention and the right in limited circumstances to require the patent owner to license to others on reasonable terms as provided by the terms of Grant No. DMR-0305147 awarded by the National Science Foundation and Grant No. RAB2CF awarded by the Armed Forces Radiobiology Research Institute, Uniformed Services University of the Health Sciences of the United States Department of Defense.

### TECHNICAL FIELD OF THE INVENTION

The present invention pertains to an optical method for selectively inactivating pathogenic microorganisms while leaving mammalian surrounding cells unharmed.

### BACKGROUND

Biochemical and pharmaceutical methods currently used for the inactivation of viral and bacterial particles, although quite successful, encounter problems of drug resistance in the target virus and bacterium. In addition, these methods have shown clinical side effects such as headache, diarrhea, and skin rash. An ultraviolet (UV) disinfection method can be used for diminishing microorganisms (Lagunas-Solar, et al., U.S. Pat. No. 6,329,136; Anderle et al., US patent No: US20060045796A1). UV lamps target both the nuclei acids (Sutherland et al., Radiation Research, Vol. 86, 3990410 (1981)) and proteins (Rosenheck et al., Proc Natl. Acad. Sci. USA. 47(11): 1775-1785 (1961)), and as a result they not only damage the viral and bacterial particles but also harm the mammalian cells and therefore have no selectivity. Also, ultraviolet irradiation raises concerns of genetic mutation. Using an intense far-infrared absorption technique (for example, a CO₂ laser, Pratt, Jr. et al., U.S. Pat. No. 4,115,280) has been proposed to alter the structure of a microorganism by exciting vibrational and/or rotational modes. However, this method also lacks selectivity as it heats up the surroundings of the biological system because water which absorbs far-infrared radiation, coexists with microorganisms in a biological system.

A nonlinear method involving four-wave mixing has been proposed (Zanni et al., US patent No: US20060063188A1) to identify and characterize molecular interactions. The method may be used for the inactivation of microorganisms; however, because it primarily targets the covalent bonds of the molecules such as stretching modes of C═O and C—C—C which usually exist in both the microorganisms and mammalian cells, it will inactivate both the pathogenic microorganisms and mammalian cells; as a result this method does not have the property of selective inactivation. Recently, a photochemical technique (Bryant et al, Arch. Pathol. Lab. Med. 131, 719-733 (2007)) has been developed to sterilize plasma using UVA light and some psoralens (UV sensitive substance that binds permanently to DNA, thereby preventing DNA replication). Such a system is currently in use in Europe on a small scale and is only used for non-cellular products such as fresh frozen plasma. The use of psoralens and UV light on platelets has caused platelet activation and destruction. The use of such technology in red cells has failed since the penetration of ultraviolet light into a bag of red cells is limited. Furthermore, a step that removes unbound psoralens from the product bag is required, since psoralen is toxic to the skin and causes severe sunburns and blindness in patients who receive psoralen and are exposed to natural UV light from the sun. There have been other proposals that employed pulsed lasers to kill unwanted microorganisms using pulsed laser irradiation in the literature. These methods, which use lasers having pulse widths in the millisecond or microsecond, or nanosecond or picosecond time scales, can inactivate harmful microorganisms; however, because very high laser intensity has to be used for inactivation, they will also damage sensitive materials such as mammalian cells. Therefore, these pulsed laser methods also lack selectivity.

The methods mentioned above inactivate microorganisms but none of them provide selectivity, namely, the ability to inactivate the pathogenic or unwanted microorganisms such as viruses, bacteria, etc. while leaving the sensitive materials like mammalian cells unharmed. Therefore, there has been a long felt but unresolved need for a method that selectively inactivates pathogenic microorganisms while leaving mammalian cells unharmed.

### REFERENCES CITED

1. K. Rosenheck, and P. Doty, The far ultraviolet absorption spectra of polypeptide and protein solutions and their dependence on conformation. Proc Natl. Acad. Sci. USA. 47(11): 1775-1785 (1961).
2. J. C. Sutherland, and K. P. Griffin, Absorption spectrum of DNA for wavelengths greater than 300 nm, Radiation Research, Vol. 86, 3990410 (1981).
3. Heinz Anderle, Peter Matthiessen, Hans-Peter Schwarz, Peter Turecek, Thomas Krell and Daniel R. Boggs, Methods for the inactivation of microorganisms in biological fluids, flow through reactors and methods of controlling the light sum dose to effectively inactivate microorganisms in batch reactors. US patent No: US20060045796A1.
4. George W. Pratt, Jr. Apparatus for altering the biological and chemical activity of molecular species. U.S. Pat. No. 4,115,280.
5. Martin T. Zanni, John C. Wright, Eric C. Fulmer, Nonlinear spectroscopic methods for identifying and characterizing molecular interactions. US patent No: US20060063188A1.
6. B. J. Bryant, and H. G. Klein, Pathogen Inactivation: The Definitive Safeguard for the Blood Supply, Arch. Pathol. Lab. Med. 131, 719-733 (2007).
7. E. C. Dykeman, O. F. Sankey, and K.-T. Tsen, Raman intensity and spectra predictions for cylindrical viruses, Physical Review E 76, 011906 (2007).
8. E. C. Dykeman and O. F. Sankey, Low Frequency Mechanical Modes of Viral Capsids: An atomistic Approach, Physical Review Letters 100, 028101 (2008).
9. E. C. Dykeman and O. F. Sankey, Theory of the low frequency mechanical modes and Raman spectra of the M13 bacteriophage capsid with atomic detail, Journal of Physics: Condensed Matter 21, 035116, (2009).
10. Y-X Yan, E. B. Gamble, Jr. and Keith A. Nelson, Impulsive stimulated scattering: General importance in femtosecond laser pulse interactions with matter, and spectroscopic applications, J. Chem. Phys. 83, 5391-5399 (1985).
11. K. A. Nelson, R. J. D. Miller, D. R. Lutz, and M. D. Fayer, Optical generation of tunable ultrasonic waves, J. Appl. Phys. 53, 1144-1149 (1982).
12. S. De Silvestri, J. G. Fugimoto, E. P. Ippen, E. B. Gamble, Jr., L. R. Williams, and K. A. Nelson, Femtosecond time-resolved measurements of optic phonon dephasing by impulsive stimulated raman scattering in α-perylene crystal from 20 to 300 K, Chem. Phys. Lett. 116, 146-152 (1985).
13. K. A. Nelson, Stimulated Brillouin scattering and optical excitation of coherent shear Waves, J. Appl. Phys. 53, 6060-6063 (1982).
14. K T Tsen, S W D Tsen, C L Chang, C F Hung, T C Wu, J G Kiang, Inactivation of Viruses by coherent excitations with a low power visible femtosecond Laser, Virology Journal 4, 50-1/6 (2007).
15. K Tsen, S-W D Tsen, O F Sankey and J G Kiang, Selective inactivation of micro-organisms with near-infrared femtosecond laser, Journal of Physics Condensed Matter 19, 472201-1/7 (2007).
16. K T Tsen, Shaw-Wei D Tsen, Chih-Long Chang, Chien-Fu Hung, T-C Wu, and Juliann G Kiang, Inactivation of viruses with a very low power visible femtosecond laser, Journal of Physics Condensed Matter 19, 322102-1/9 (2007).
17. Kong-Thon Tsen, Shaw-Wei D. Tsen, Chih-Long Chang, Chien-Fu Hung, T.-C. Wu, and Juliann G. Kiang, Inactivation of viruses by laser-driven coherent excitations via impulsive stimulated Raman scattering process, Journal of Biomedical Optics 12, 064030 (2007).
18. K T Tsen, S-W D Tsen, C-F Hung, T-C Wu and J. G Kiang, Selective inactivation of human immunodeficiency virus with subpicosecond near-infrared laser pulses, Journal of Physics Condensed Matter 20, 252205-1/4 (2008).
19. Constructions and detailed protocols for the preparation of the pseudovirions can be found online at http://home.ccr.cancer.gov/lco/default.asp.
20. H. D. Wang et al. Glutaraldehyde modified mica: A new surface for atomic force microscopy of chromatin. Biophysical Journal 83, 3619-3625 (2002).
21. X. Ji, J. Oh, A. K. Dunker, and K. W. Hipps, Effects of relative humidity and applied force on atomic force microscopy images of the filamentous phage fd. Ultramicroscopy, 72, 165-176 (1998).
22. Ki-Tae Nam, Beau R. Peelle, Seung-Wuk Lee, and Angela M. Belcher, Genetically Driven Assembly of Nanorings Based on the M13 Virus. Nano Letters, 4, 23-27 (2004).
23. D. Anselmetti, R. Luthi, E. Meyer, T. Richmond, M. Dreier, J. E. Frommer, and H. J. Guntherodt, Attractive-mode imaging of biological materials with dynamic force microscopy. Nanotechnology 5, 87-94 (1994).
24. Lagunas-Solar, et al., Method for laser inactivation of infectious agents, U.S. Pat. No. 6,329,136.

### SUMMARY OF THE INVENTION

This summary is provided to introduce a selection of concepts in a simplified form that are further described in the detailed description of the invention. This summary is not intended to identify key or essential inventive concepts of the claimed subject matter, nor is it intended for determining the scope of the claimed subject matter.

The method disclosed herein addresses the above stated need for selectively inactivating microorganisms while leaving mammalian cells unharmed. The method disclosed herein employs: (a) a light source having a wavelength transparent to water, (b) a process which produces significantly large vibrations on the outer structure of microorganisms, for example, the protein shell of a virus, through scattering and not via absorption of light, and (c) a process which targets the pathogenic microorganisms but leaves mammalian cells unharmed. The method disclosed herein accomplishes these goals through proper manipulation and control of femtosecond pulsed lasers via an impulsive stimulated Raman scattering (ISRS) process. The ISRS process produces severe damage to the outer structures of pathogenic microorganisms while leaving sensitive materials, for example, mammalian cells, unharmed.

The method for selectively inactivating microorganisms while leaving mammalian cells unharmed disclosed herein, comprises: exciting the microorganisms in a fluid and/or a tissue into vibrational states with a single femtosecond laser beam of radiation at a wavelength that is in a range of an electromagnetic spectrum where water is substantially transparent, wherein the vibrational states of the excited microorganisms are high amplitude, low-frequency acoustic vibrations on an outer structure of the microorganisms that diminish the activity of the microorganisms.

The method disclosed herein targets the mechanical property of an outer structure of the microorganism, for example, the protein coat of a virus. The method disclosed herein targets the weak links, for example, the hydrogen bonds and hydrophobic bonds, on the outer structure of the microorganisms. By properly manipulating and controlling the laser parameters, for example, wavelength, pulse width, repetition rate and power density of a femtosecond laser system, the method disclosed herein inactivates harmful microorganisms and leaves the mammalian cells unharmed.

The method disclosed herein is, for example, used for cleansing blood components, disinfecting drinking water, treating viral and bacterial diseases, extracting nucleic acid from microorganisms, manufacturing vaccines, etc. These and other advantages of the method disclosed herein, as well as additional features, will be apparent from the following detailed description.

### BRIEF DESCRIPTION OF THE DRAWINGS

The foregoing summary, as well as the following detailed description of the invention, is better understood when read in conjunction with the appended drawings. For the purpose of illustrating the invention, exemplary constructions of the invention are shown in the drawings. However, the invention is not limited to the specific methods and instrumentalities disclosed herein. In the drawings, like reference numbers refer to like elements or acts throughout the drawings.

FIG. 1 illustrates a method for selectively inactivating microorganisms while leaving mammalian cells unharmed.

FIGS. 2A-2C exemplarily illustrate schematics showing how a M13 bacteriophage is inactivated by a femtosecond pulsed laser.

FIG. 3 exemplarily illustrates a system for inactivating M13 bacteriophages.

FIG. 4A exemplarily illustrates a graphical representation showing activity of three assays for a sample with about 1×10³ plaque forming units (pfu) of M13 bacteriophages without laser irradiation.

FIG. 4B exemplarily illustrates a graphical representation showing activity of three assays for a sample with about 1×10³ plaque forming units (pfu) of M13 bacteriophages after laser irradiation by a visible femtosecond laser for about 10 hours.

FIG. 5 exemplarily illustrates a graphical representation showing results of plaque forming assays on a M13 bacteriophage sample at a titer of 1×10³ plaque forming units (pfu) as a function of the excitation laser power density of a visible femtosecond laser.

FIG. 6A exemplarily illustrates a graphical representation showing inactivation of a M13 bacteriophage sample with a near-infrared sub-picosecond fiber laser.

FIG. 6B exemplarily illustrates a graphical representation showing inactivation of a tobacco mosaic virus sample with a near-infrared sub-picosecond fiber laser.

FIG. 6C exemplarily illustrates a graphical representation showing inactivation of a human papillomavirus sample with a near-infrared sub-picosecond fiber laser.

FIG. 6D exemplarily illustrates a graphical representation showing inactivation of a human immunodeficiency virus sample with a near-infrared sub-picosecond fiber laser.

FIGS. 7A-7B exemplarily illustrate atomic force microscope images of a M13 bacteriophage sample without laser irradiation and a M13 bacteriophage sample with laser irradiation by a near-infrared sub-picosecond fiber laser, respectively.

FIGS. 7C-7D exemplarily illustrate atomic force microscope images of a tobacco mosaic virus sample without laser treatment and a tobacco mosaic virus sample with laser-irradiation by a near-infrared sub-picosecond fiber laser, respectively.

FIG. 8 exemplarily illustrates a graphical representation showing number of plaques for a M13 bacteriophage sample as a function of the exposure time to radiation by a near-infrared sub-picosecond fiber laser.

FIG. 9 exemplarily illustrates a graphical representation showing number of plaques for a M13 bacteriophage sample as a function of the excitation laser power density of a near-infrared sub-picosecond fiber laser.

FIG. 10 exemplarily illustrates a graphical representation showing two assays showing the number of salmonella bacteria for control and laser irradiated samples by a visible femtosecond laser as indicated.

FIG. 11 exemplarily illustrates a graphical representation showing two typical assays showing the number of E-coli bacteria for control and laser irradiated samples by a visible femtosecond laser as indicated.

FIG. 12 exemplarily illustrates a table showing dependence of inactivation of M13 bacteriophages on the pulse width of a visible pulsed laser.

FIG. 13 exemplarily illustrates a table showing threshold laser power density for the inactivation of a variety of viruses and cells by a near-infrared sub-picosecond fiber laser.

### DETAILED DESCRIPTION OF THE INVENTION

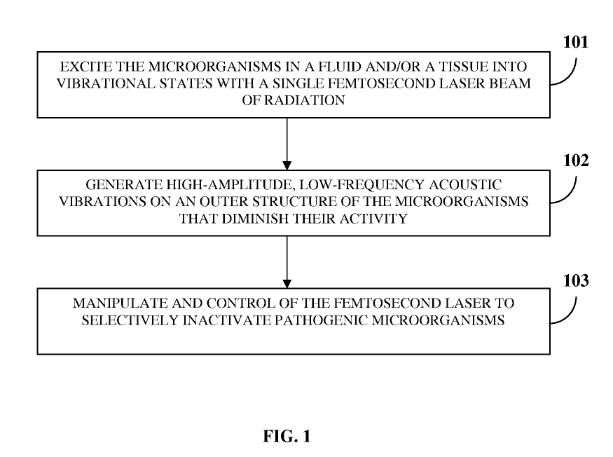

FIG. 1 illustrates a method for selectively inactivating microorganisms while leaving mammalian cells unharmed. The microorganisms, for example, viruses, bacteria and protozoa, are excited 101 in a fluid and/or a tissue into vibrational states with a single femtosecond laser beam of radiation at a wavelength that is in a range of an electromagnetic spectrum to which water is substantially transparent. The fluid is, for example, water, whole blood, blood components in their buffer solutions, etc. The electromagnetic spectrum to which the water is substantially transparent covers a range of electromagnetic waves with a wavelength, for example, from near-infrared to visible spectrum, or from about 400 nanometers to about 1.3 micrometers. The excitation of the microorganisms results from an impulsive stimulated Raman scattering (ISRS) process. High-amplitude, low-frequency acoustic vibrations are generated 102 on an outer structure of the microorganisms due to the laser excitation which diminish the activity of the microorganisms. The high-amplitude, low-frequency acoustic vibrations correspond to vibrational frequency in the range, for example, from about 1 gigahertz (GHz) to about 1000 gigahertz (GHz). The vibrational states of the excited microorganisms are low-frequency, acoustic vibrational states of the outer structure of the microorganisms. The outer structure of the microorganisms is, for example, a protein shell of a virus, a lipid bi-layer of a bacterium, etc. Manipulation and control 103 of the femtosecond laser enable selective inactivation of pathogenic microorganisms while leaving the surrounding mammalian cells unharmed. The single femtosecond laser beam is a single laser beam produced, for example, by a continuous wave (CW) mode-locked titanium-sapphire (Ti-sapphire) laser, a pulsed fiber laser, or an amplifier laser system on which a CW mode-locked laser is based, with pulse width that is less than one picosecond. The manipulation and control of the femtosecond laser comprises properly choosing pulse width, wavelength, power density and repetition rate of the femtosecond laser.

The method disclosed herein inactivates viral/bacterial particles by mechanical means and with selectivity. The method disclosed herein targets the outer structure of the microorganism, a paradigm shift from chemical or biological treatments, and is capable of inactivating the unwanted viruses/bacteria while leaving the sensitive materials, for example, mammalian cells unharmed. The method disclosed herein uses femtosecond laser technology to coherently excite large amplitude vibrations on the outer structure of microorganism, for example, a protein shell of a virus, through an impulsive stimulated Raman scattering (ISRS) process, which damages the protein coat/lipid bi-layer of the microorganisms and leads to the inactivation of the microorganisms.

The microorganisms can be inactivated by femtosecond laser pulses through the ISRS process. For a continuous wave (CW) laser or light source, inactivation of microorganisms such as viruses through the proposed ISRS process will not work. This is because the impulsive force provided by the light should last no longer than a quarter of the oscillation period of the relevant vibrational mode on the outer structure of a microorganism in order to achieve an efficient excitation of a large-amplitude vibrational mode. The effect is like giving a child a push on a swing. If the pushing force is constant, then the maximum amplitude is achieved when the force is applied for one-quarter of a cycle of the swing. A CW laser would be like pushing the child all the time and as a result, no amplitude of vibration is achieved.

Consider an example for viruses. Viruses have frequencies of oscillation for the global motion of the viral capsid (e.g., the outer structure) that have recently been computed (Dykeman et al., Physical Review E 76, 011906 (2007); Dykeman et al., Physical Review Letters 100, 028101 (2008). Dykeman et al., Journal of Physics: Condensed Matter 21, 035116, (2009)), to be of the order of 30 gigahertz (GHz) or 1 cm⁻¹ in spectroscopic terms. This is in the microwave range. Directly exciting these oscillations with microwave radiation is problematic since water, which usually coexists with microorganisms in a biological system, absorbs microwaves in this spectral range and heats up everything in the system indiscriminately. However, water is transparent to visible light or near-infrared light. Therefore, electromagnetic radiation at such a range of wavelengths is the most suitable light source for exciting the microorganisms embedded in water. The electromagnetic light wave from a visible or near-infrared laser produces an electric field that alternates much faster than the vibrational frequencies of viral capsids. Therefore, direct excitation of about 30 GHz vibrations by a visible/near-infrared laser through absorption process is not possible. Instead, vibrations can be produced indirectly by exciting the virus with a pulsed laser having a pulse width that lasts no longer than a quarter of the oscillation period of the relevant vibrational mode, in this case about 30 GHz, on the outer structure of a virus. This “timed kick” of an object through an ultrashort pulse is known as the impulsive stimulated Raman scattering (ISRS) process (Yan et al., J. Chem. Phys. 83, 5391-5399 (1985); Nelson et al., J. Appl. Phys. 53, 1144-1149 (1982); De Silvestri et al., Chem. Phys. Lett. 116, 146-152 (1985); Nelson, J. Appl. Phys. 53, 6060-6063 (1982); Tsen et al., Virology Journal 4, 50-1/6 (2007); Tsen et al., Journal of Physics: Condensed Matter 19, 472201-1/7 (2007); Tsen et al., Journal of Physics Condensed Matter 19, 322102-1/9 (2007); Tsen et al., Journal of Biomedical Optics 12, 064030 (2007); Tsen et al., Journal of Physics Condensed Matter 20, 252205-1/4 (2008)). By choosing the pulse duration to be near or shorter than the oscillation period of the normal mode of the viral particle, the laser pulse has significant spectral content at the Stokes-shifted frequency necessary to bring the outer structure, for example, the outer protein shell of a viral particle into oscillation.

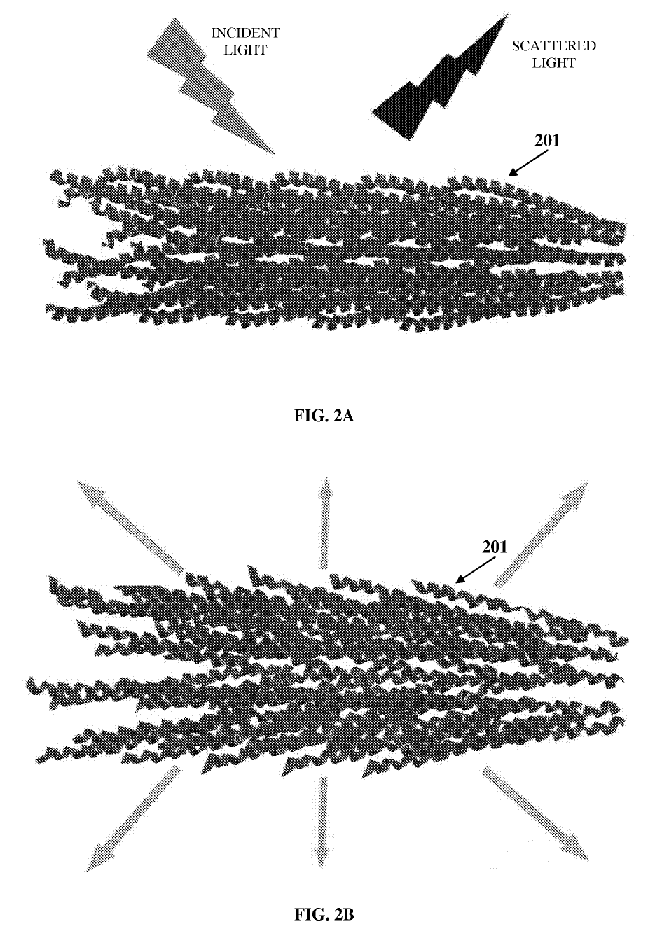

FIGS. 2A-2C exemplarily illustrate schematics showing how a M13 bacteriophage 201 is inactivated by a femtosecond pulsed laser. In FIGS. 2A-2C, the single-strained deoxyribonucleic acid (DNA) of the M13 bacteriophage is not shown for the sake of simplicity. The output of the second harmonics of a CW mode-locked Ti-sapphire laser is used for the irradiation. The M13 bacteriophage 201 is a virus that only infects Escherichia coli (E-coli) bacteria and is in the form of a long tube. The outer structure of a M13 bacteriophage 201 is composed of α-helix proteins. The electric field from about a 100 femtosecond laser pulse produces an impulsive force through the induced charge polarization on the virus, as illustrated in FIG. 2A. The laser scatters off the M13 bacteriophage 201. This mechanical impact coherently excites Raman-active vibrational modes on the capsid of the virus. The impulsive force from the laser sets the outer structure or the protein shell of M13 bacteriophage 201 into vibrations as illustrated in FIG. 2B. Under proper laser conditions, that is, if the pulse width and spectral width and intensity of the femtosecond laser are appropriately chosen, the vibrational modes can be excited to such high energy states as to break off the weak links on the capsid of the virus as exemplarily illustrated in FIG. 2C, thereby damaging or disintegrating the capsid and leading to the inactivation of the virus.

The ISRS process excited by a femtosecond pulsed laser destroys harmful microorganisms while sparing the mammalian cells. For a single pulsed laser beam to inactivate the harmful microorganisms via the ISRS process, the full-width at the half-maximum (FWHM) of the spectral width of pulsed laser beam should be larger than the vibrational energy of the microorganisms. Since the vibrational energy of the outer protein shell of the harmful microorganisms is typically of the order of 10 GHz, that is, since the vibrational energy of viruses lies, for example, between 30 GHz and 500 GHz, for a transform-limited pulsed laser, the pulse width has to be shorter than 1 picosecond in order for it to inactivate the harmful microorganisms through ISRS process. In a transform-limited pulsed laser, ΔE·Δt≅ [symbol] where ΔE is the full-width at half maximum (FWHM) of the spread of the laser energy; Δt is the FWHM of the laser pulse width and [symbol] ≡h/2π, where h is Planck's constant.

On the other hand, the physical size effects of different microorganisms can be used to explain why the selective inactivation can work with the ISRS process excited by a femtosecond pulsed laser system. Viral and bacterial particles are typically much smaller than the mammalian cells. For example, the human immunodeficiency virus (HIV) is an enveloped virus with a capsid and is about 0.1 μm in diameter; whereas the shape of a human red blood cell is like a donut and is about 10 μm in diameter and 2 μm in thickness. The mouse dendritic cell is about 10 μm in diameter. Since the viruses and cells are embedded in water, the water molecules will damp the vibrations excited by the laser. The relatively large size of either the human red blood cell or the mouse dendritic cell as compared with that of the viral particle means that there are more water molecules surrounding the red blood cells and dendritic cells than HIV. The damping associated with the coherent/incoherent excitation created by the laser is less for HIV than for red blood cells or dendritic cells. As a result, the amplitude of vibrations created on the outer structures by a given laser power density can be much higher for pathogenic microorganism such as HIV than for mammalian cells like red blood cells or mouse dendritic cells.

The following examples elucidate some of the features of the method disclosed herein. As these examples are presented for illustrative purposes, they should not be used to construe the scope of the method disclosed herein in a limited manner, but rather should be considered as expanding the foregoing description of the invention as a whole.

### Example 1

This example demonstrates that by using a very low power (as low as 0.5 nj/pulse) visible femtosecond laser having a wavelength of 425 nanometers (nm) and a pulse width of 100 femtosecond (fs), M13 bacteriophages 201 are inactivated when the laser power density was greater than or equal to 50 MW/cm². The inactivation of M13 bacteriophages 201 is determined by plaque counts and is found to depend on the pulse width as well as the power density of the excitation laser, which are consistent with predictions from the ISRS process.

The M13 bacteriophage 201 samples used in this work were purchased from Stratagene® Corporation, La Jolla, Calif., U.S.A.

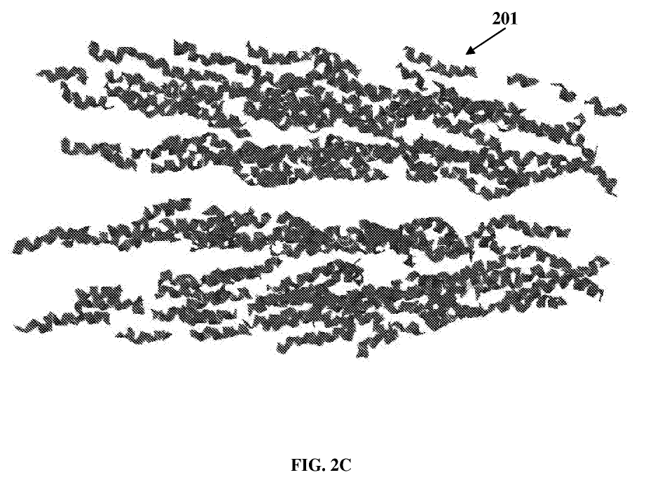

FIG. 3 exemplarily illustrates a system for inactivating M13 bacteriophages. The system disclosed herein comprises a diode-pumped CW mode-locked Ti-sapphire laser 301, a harmonic generator 302, mirrors 303, and a microscope objective (M.O.) 304. The diode-pumped CW mode-locked Ti-sapphire laser 301 comprises a diode laser and a CW mode-locked Ti-sapphire laser. The diode laser pumps the CW mode-locked Ti-sapphire laser. The diode laser was purchased from Coherent Inc., Santa Clara, Calif., U.S.A. The diode laser provides 5 watts of continuous wave, linearly polarized laser beam at a wavelength of 532 nm. The CW mode-locked Ti-sapphire laser was purchased from Del Mar Photonics, San Diego, Calif., U.S.A. The diode-pumped CW mode-locked Ti-sapphire laser 301 produced a 60 fs, almost transform-limited laser pulse train at a repetition rate of about 80 MHz, and a central wavelength of about 850 nm. The Harmonic generator 302 was purchased from Del Mar Photonics, San Diego, Calif., U.S.A. The harmonic generator 302 is equipped with a BBO (β-barium borate) nonlinear crystal. The harmonic generator 302 was used to convert the fundamental near-infrared wavelength at 850 nm from the CW mode-locked Ti-sapphire laser to the visible wavelength at 425 nm. The pulse width and typical average power from the harmonic generator 302 is about 100 fs and 150 mW, respectively. The microscope objective 304 is a Mitutoyo Infinity-corrected long working distance objective with a working distance of about 2 cm, purchased from Edmund Optics Inc., Barrington, N.J., U.S.A. The mirrors 303 are for example, dielectric mirrors 303, purchased from THORLABS, Newton, N.J., U.S.A.

The diode-pumped CW mode-locked Ti-sapphire laser 301 is the excitation source employed in the system. The laser 301 produces a continuous train of 60 fs pulses at a repetition rate of 80 megahertz (MHz). As illustrated in FIG. 3, the output of the second harmonic generation system (SHG) or the harmonic generator 302 of the Ti-sapphire laser 301 is used to irradiate the sample (S) 305. The magnification shows the sample 305 area where the laser beam is tightly focused. The cylindrical volume where the laser beam focuses most tightly defines the active volume for the inactivation of M13 bacteriophages through the ISRS process. The excitation laser 301 is chosen to operate at a wavelength of λ=425 nm and with an average power of about 40 milliwatts (mW) unless otherwise specified. The laser 301 has a pulse width of full-width at half maximum (FWHM)≅100 fs. A lens of extra long focus length is used to focus the laser beam into the sample 305 area. The laser illuminated volume defines the active volume for the interaction of the laser 301 with the M13 bacteriophage through the ISRS process, In order to facilitate the interaction of the laser 301 with the M13 bacteriophages which are inside a glass cuvette and diluted in 0.1 ml water, a magnetic stirrer 306 is set up so that M13 bacteriophages enter the laser-focused volume as described above and interact with the photons. The magnetic stirrer 306 is used to facilitate the interaction of photons with the microorganisms within the vials. The magnetic stirrer 306 is for example Model: PC-420, available from Corning, N.Y., U.S.A. The vials contain the microorganism in its buffer solution. The vials, for example, are glass vials. The glass vials were purchased from VWR International Inc., West Chester, Pa., U.S.A. The laser-irradiated M13 bacteriophage samples 305 contain 1×10⁷ pfu/ml, where pfu is plaque forming units. Plaque forming units is a measure of the number of particles capable of forming plaques per unit volume, such as virus particles. The assays were performed on the laser-irradiated samples 305 after proper dilution. The typical exposure time of the sample 305 to laser irradiation was about 10 hours. The amount of time, for example, 10 hours, required reflects the particular arrangement of the system and is not related to the efficiency of the inactivation of M13 bacteriophages by the laser system 301. Preliminary results indicated that a more efficient mixing arrangement resulted in a much shorter time required for the observation of inactivation of the M13 bacteriophages. A thermal couple is used to monitor the temperature of the sample 305 to ensure that the results are not due to the heating effects. The increase of the temperature of the M13 bacteriophage samples 305 is found to be less than 3° C. after 10 hours of laser irradiation. The experimental results disclosed herein are obtained at T=25° C. and with the single-laser-beam excitation.

The activity of M13 bacteriophages is determined by plaque counts. In brief, M13 bacteriophages with nominally prepared 1×10³ pfu are added into a tube of soft agar at 70° C. containing 0.3 ml of bacteria culture. As used herein, the term “nominally prepared” refers to preparation/dilution of the M13 bacteriophage samples 305 based on the pfu concentration specified by the manufacturer upon purchasing. The mixture is mixed well by vortexing and then poured onto a luria broth (LB) agar plate immediately. The plate was swirled well in order to spread the mixture over the entire plate evenly. The mixture on the agar plate was incubated for 8-16 hr. The plaques formed on the plate were counted.

The data is expressed as mean±SD. Student's t-test was used for comparison of group with 5% as significant level.

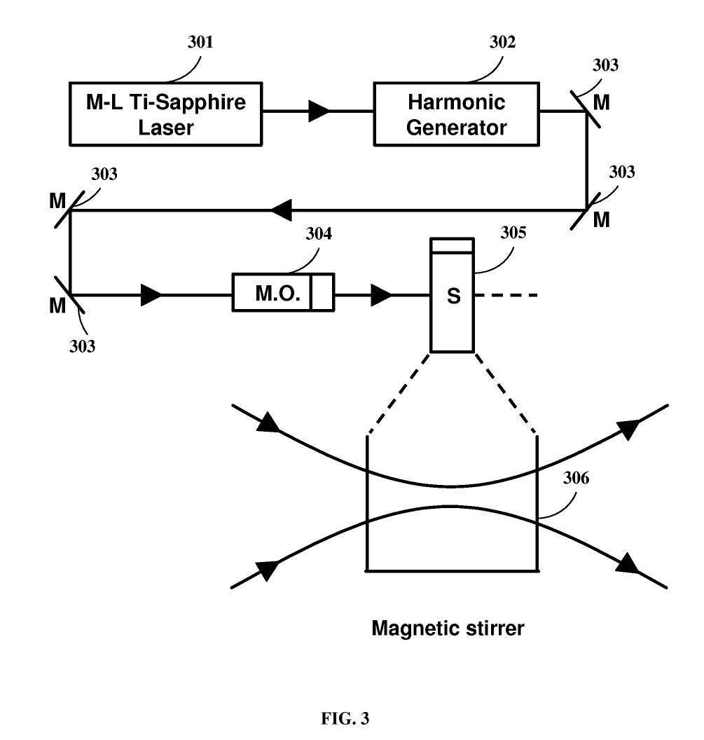

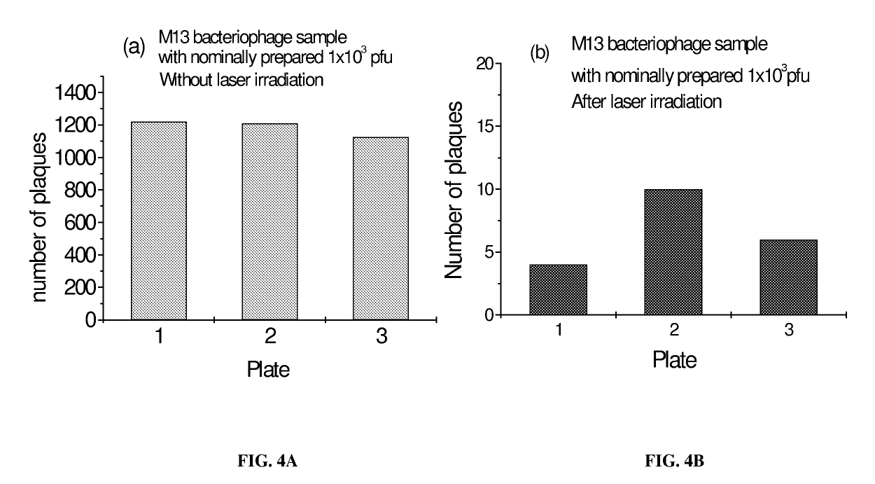

FIG. 4A exemplarily illustrates a graphical representation showing activity of three assays for a sample with nominally prepared 1×10³ plaque forming units (pfu) of M13 bacteriophages without laser irradiation. The number of plaques is determined to be 1184±52 counts. FIG. 4B shows the corresponding runs after the laser irradiation by a visible femtosecond laser for about 10 hours. As illustrated in FIG. 4B, the number of plaques after laser irradiation is 7±3 counts. It is seen that there is a minimal amount of plaques for the laser irradiated samples as compared with the reference samples, indicative of the inactivation of M13 bacteriophages by the laser irradiation. The observed inactivation of M13 bacteriophages is attributed to laser-driven coherent excitations through the ISRS process.

ISRS has been successfully demonstrated in molecular as well as solid state systems (see Yan et al., J. Chem. Phys. 83, 5391-5399 (1985); Nelson et al., J. Appl. Phys. 53, 1144-1149 (1982); De Silvestri et al., Chem. Phys. Lett. 116, 146-152 (1985); Nelson, J. Appl. Phys. 53, 6060-6063 (1982); Tsen et al., Virology Journal 4, 50-1/6 (2007); Tsen et al., Journal of Physics: Condensed Matter 19, 472201-1/7 (2007); Tsen et al., Journal of Physics Condensed Matter 19, 322102-1/9 (2007); Tsen et al., Journal of Biomedical Optics 12, 064030 (2007); and Tsen et al., Journal of Physics Condensed Matter 20, 252205-1/4 (2008)). The ISRS process is used to selectively inactivate microorganisms when excited by a properly manipulated and controlled femtosecond pulsed laser. For a single-laser-beam excitation, if the damping is ignored, the amplitude (R₀) of the displacement away from the equilibrium intermolecular distance caused by the ISRS can be shown to be given by equation (1) (Yan et al., J. Chem. Phys. 83, 5391-5399 (1985)) below:

R₀=4πI(δα/δR)₀e^{-ω}^{₀}^{²}^{τ}^{_{L}}^{^{2/4}}/mω₀nc (1)

where I is the intensity of the excitation laser; α is the polarizability of the medium; R is the displacement away from the equilibrium intermolecular distance; δα/δR is proportional to the Raman scattering cross section; ω₀ is the angular frequency of the excited coherent vibrational mode; τ_{L} is the FWHM of the pulse width of the excitation laser; m is the molecular mass; n is the index of refraction; and c is the speed of light.

For the one-laser-beam excitation experiment, the primary beam as well as the Stokes beam, whose photon energies are denoted by ω_{L} and ωₛ, respectively, define the excited coherent vibrations with energy [symbol] such that [symbol] = [symbol] − [symbol] . As a result, the FWHM of the spectral width of the excitation laser has to be larger than the energy of the excited coherent vibrations, which, because of the Gaussian distribution of the excitation laser in both time and space and by using uncertainty principle, gives rise to the factor: e^{-ω}^{₀}^{²}^{τ}^{_{L}}^{^{2/4}}

in equation (1). This exponential dependence indicates that the product of angular frequency of the excited coherent vibration (ω₀) and the FWHM of the excitation pulse width (τ_{L}) has to be small in order that the amplitude R₀ of the excited coherent vibration can be significant, that is, ω₀τ_{L}≧1. This explains why the excitation laser should be ultrashort in pulse width, e.g., shorter than 1 picosecond (ps) for the ISRS to work.

From equation (1), it is clear that larger Raman cross sections, higher laser power densities, as well as lower vibrational frequencies, contribute to bigger excited vibrational amplitude. For a moderate Raman scattering cross section, a sufficiently low vibrational frequency and a reasonable excitation power density, the amplitude of the vibrational displacement in the 0.01 to 1 Å could be achieved through ISRS.

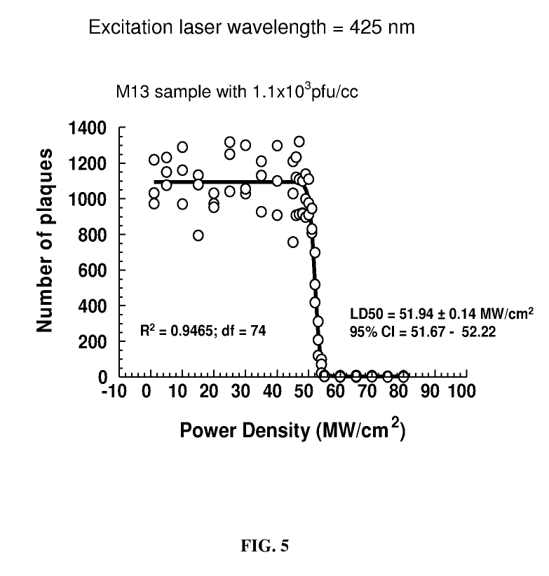

FIG. 5 exemplarily illustrates a graphical representation showing results of plaque forming assays on a M13 bacteriophage sample at a titer of 1×10³ pfu as a function of the excitation laser power density of a visible femtosecond laser. FIG. 5 shows the number of plaques as a function of the laser power density for M13 bacteriophage samples with 1.1×10³ pfu after being irradiated with an excitation laser having 100 fs-pulse width and λ=425 nm. The output of the second harmonics generation system of a CW mode-locked Ti-sapphire laser is used for the irradiation. An abrupt inactivation of the M13 bacteriophages at an excitation laser power density of about 50 MW/cm² is observed. This observation suggests that the M13 bacteriophages become inactivated as the amplitude of the vibrations exceeds a certain threshold. The few number of plaques observed in the irradiated sample is a manifestation of almost complete inactivation of the M13 bacteriophages in the sample.

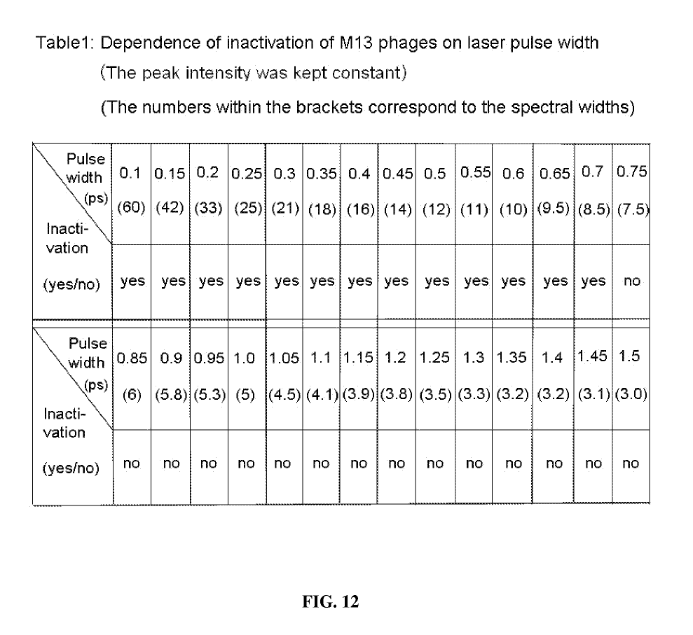

It is also observed that within the statistical error of the experiments, there is no observable inactivation of the M13 bacteriophages if the pulse width of the excitation laser is longer than about 800 fs while the intensity of the excitation laser remains constant at ≅6.4×10⁻⁶ J/cm². The experimental results are summarized in the table illustrated in FIG. 12. According to equation (1) above, if the laser intensity remains constant, the amplitude of vibrational displacement excited by an ultrashort laser decreases with the increasing laser pulse width. The experimental results in the table illustrated in FIG. 12 are consistent with this prediction, suggesting that ISRS can be the physical mechanism behind the inactivation of M13 bacteriophages.

### Example 2

In this example, it is shown that the method disclosed herein can be used to selectively inactivate viral particles ranging from non-pathogenic viruses, for example, M13 bacteriophage, tobacco mosaic virus (TMV) to pathogenic viruses, for example, human papillomavirus (HPV) and human immunodeficiency virus (HIV) while leaving sensitive materials like human Jurkat T cells, human red blood cells, and mouse dendritic cells unharmed.

The excitation source used in the inactivation of viruses is a compact, ultrashort pulsed fiber laser. The experimental arrangement is similar to the system illustrated in FIG. 3 except that the CW mode-locked Ti-sapphire laser is replaced with an ultrashort pulsed fiber laser. The ultrashort pulsed fiber laser, which has a wavelength of 1.55 μm, is operated at a repetition rate of 500 kHz and 5 μJ per laser pulse. The output of the second harmonic generation system of the fiber laser is used in the laser-irradiation experiments. The second harmonic generation system has a wavelength of 776 nm, about 1.4 μJ per laser pulse, a pulse width of full-width-half-maximum of about 600 fs and a spectral width of about 70 cm⁻¹. Water which usually coexists with biological microorganisms, absorbs radiation at 1.55 μm severely, but is rather transparent in the near-infrared and visible ranges. This is the reason for the use of the second harmonic generation (SHG) system beam. In the experiments, a single-laser beam is used for the inactivation of viruses. Different laser power density is achieved by varying the average laser power and the size of the laser beam with an achromatic lens of long focal length. A magnetic stirrer 306, for example, Corning Model PC-420, is used to stir the viral sample in its buffer solution so as to facilitate the interaction of the laser with the viral particles. The duration of the laser irradiation is about 2 hours in the experiments. The laser-irradiation experiments are carried out at T=25° C. The data is expressed in the form of mean±standard deviation.

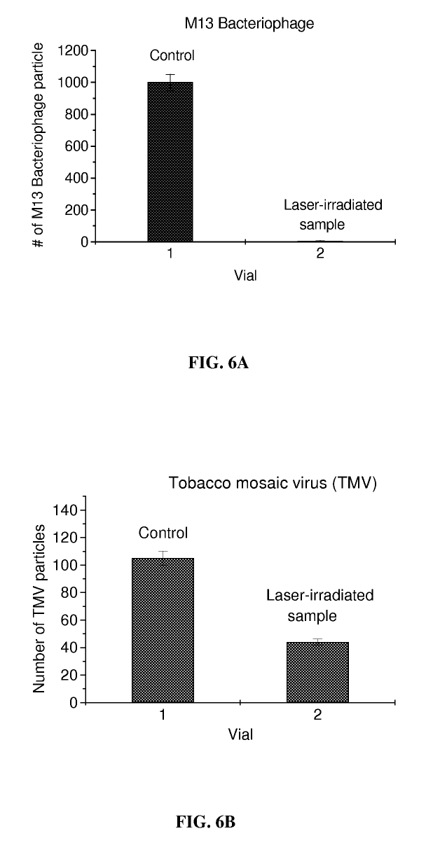

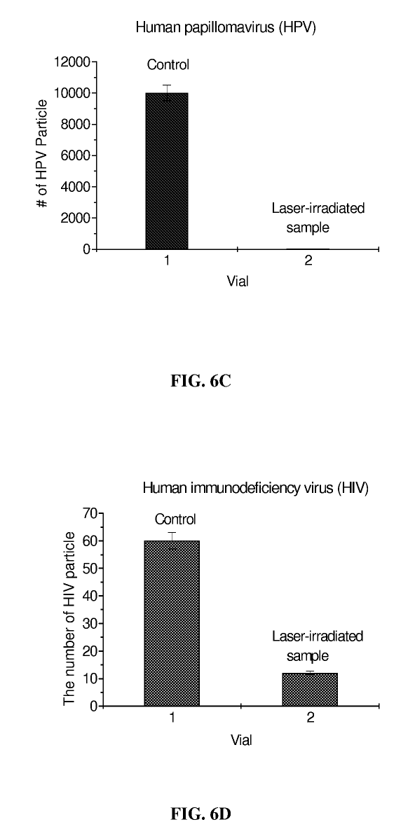

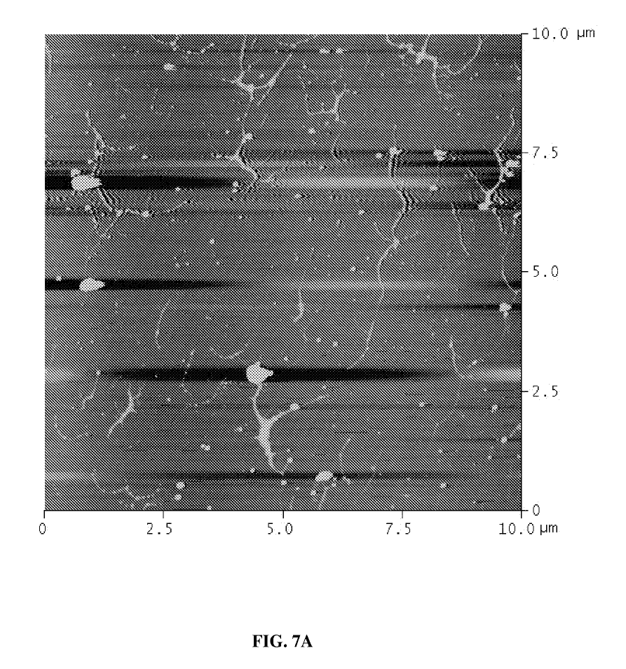

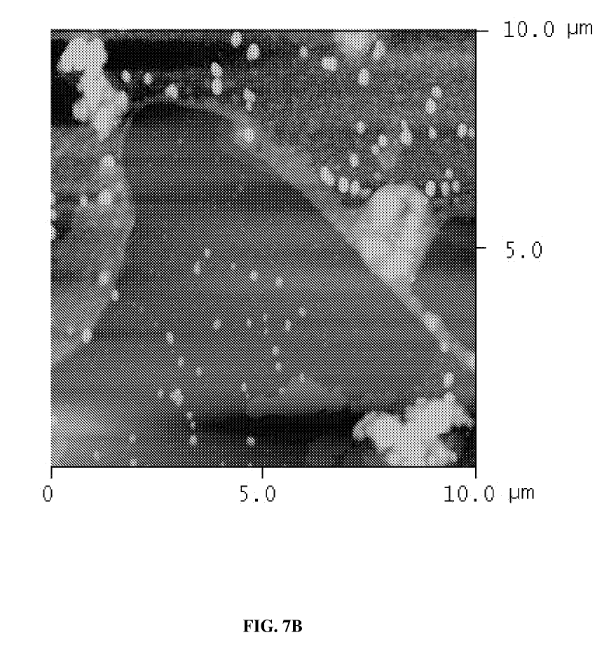

FIG. 6A exemplarily illustrates a graphical representation showing inactivation of a M13 bacteriophage sample with a near-infrared subpicosecond fiber laser. In FIGS. 6A-6D, the first vial corresponds to the control and the second one represents a laser-irradiated sample. FIG. 6A shows the number of plaques of two assays for a sample with 1×10³ pfu of M13 bacteriophages without the laser irradiation (control) and with laser-irradiation. The laser power density used is 200±20 MW/cm². The number of plaques is determined to be (990±49) counts for the control. In contrast, the number of plaques after laser irradiation is (3±2) counts. A minimal number of plaques remain after the laser irradiated sample as compared with the control, indicative of the efficient inactivation of M13 bacteriophages by the subpicosecond near-infrared fiber laser irradiation. A viral load reduction of about 10³ was observed.

FIG. 6B exemplarily illustrates a graphical representation showing inactivation of a tobacco mosaic virus (TMV) sample with a near-infrared sub-picosecond fiber laser. The assay of the TMV is performed by counting the single-stranded ribonucleic acid (RNA) released in the laser-irradiated sample with atomic force microscopy (AFM), that is, one count of the single-stranded RNA observed in the AFM image corresponds to the inactivation of one TMV in the laser irradiated sample. FIG. 6B shows the number of TMV particles in the control and laser-irradiated samples, respectively. The control has (105±6) TMV particles, whereas the laser-irradiated sample has (44±3) TMV particles. The laser power density employed is 1.0±0.1 GW/cm². The subpicosecond near-infrared fiber laser irradiation reduced the viral load by a factor of about 55%.

FIG. 6C exemplarily illustrates a graphical representation showing inactivation of a human papillomavirus (HPV) sample with a near-infrared sub-picosecond fiber laser. The inactivation of the HPV is determined from secreted alkaline phosphatase (SEAP) assays Constructions and detailed protocols for the preparation of the pseudovirions can be found online at http://home.ccr.cancer.gov/lco/default.asp. FIG. 6C shows the number of HPV particle for control and laser-irradiated samples, respectively. The control has (9980±400) HPV particles and the laser-irradiated sample has (2±1). The laser power density used is 1.0±0.1 GW/cm². A viral load reduction of about 10⁴ was recorded.

FIG. 6D exemplarily illustrates a graphical representation showing inactivation of a human immunodeficiency virus (HIV) sample with a near-infrared sub-picosecond fiber laser. The inactivation of HIV is assayed by monitoring the infectivity of U373-MAGI-CXCR4_{CEM} cells. FIG. 6D shows the number of infected cells—an indicator of the number of HIV, for control and laser-irradiated HIV samples, respectively. The laser power density used in the experiments is 1.1±0.1 GW/cm². The control sample revealed infection of (60±3) CD4⁺ T-cells; whereas the laser-irradiated sample (12±1) revealed a reduction of viral infectivity of about 80%.

In another embodiment, the method disclosed herein for the inactivation of microorganisms in water and in buffer solutions of microorganisms, may be utilized in the disinfection of microorganisms in tissue with a femtosecond laser of suitable wavelength which maximizes the penetration depth in tissue.

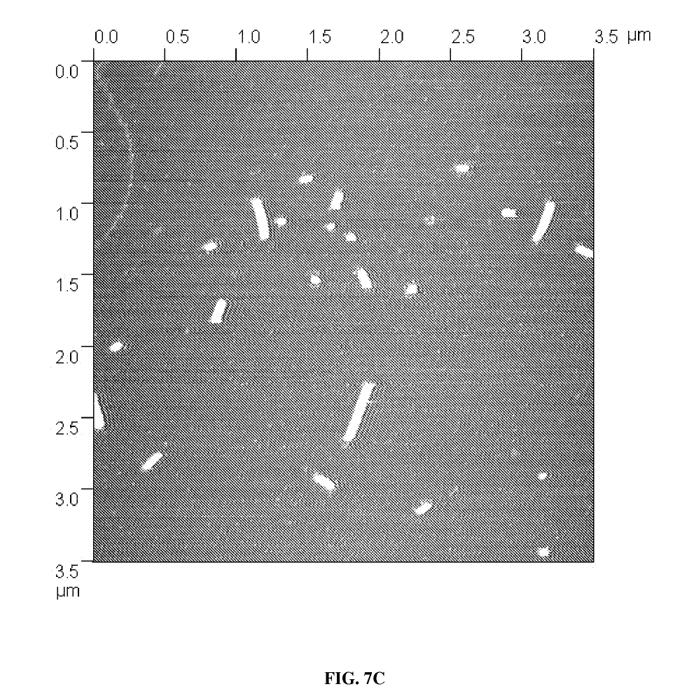

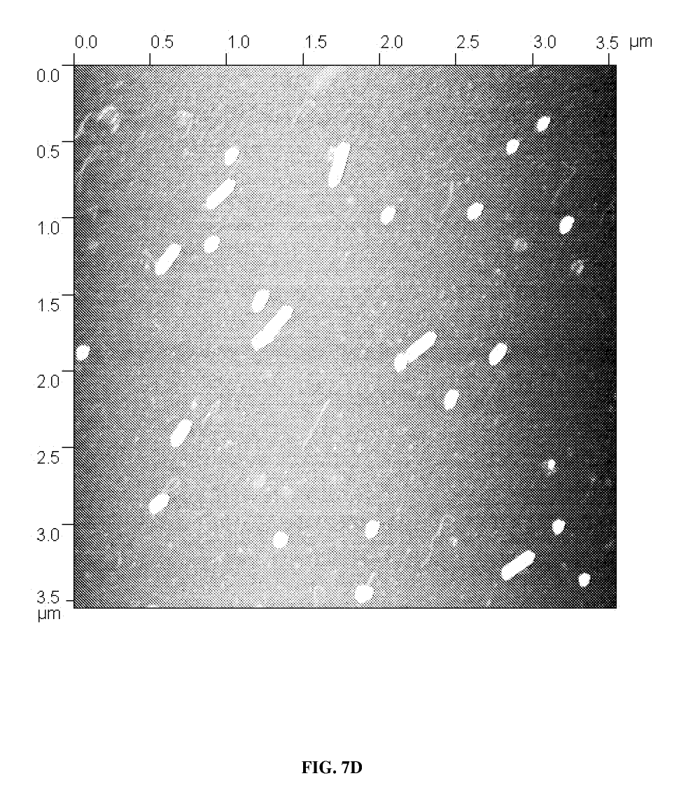

FIGS. 7A-7B exemplarily illustrate atomic force microscope (AFM) images of a M13 bacteriophage sample without laser irradiation and a M13 bacteriophage sample with laser irradiation by a near-infrared sub-picosecond fiber laser, respectively. Atomic force microscope (AFM) images of M13 bacteriophages and tobacco mosaic viruses are produced in a manner similar to that reported in literature (see for examples, Wang et al., Biophysical Journal 83, 3619-3625 (2002); Ji et al., Ultramicroscopy, 72, 165-176 (1998); Nam et al., Nano Letters, 4, 23-27 (2004); Anselmetti et al., Nanotechnology 5, 87-94 (1994)). M13 bacteriophage is a rod-shape virus with a diameter of about 6 nm and a length of about 850 nm. The capsid of the M13 bacteriophage is made up of proteins assembled in a helical shape and wrapped around a single-stranded DNA. The laser power density used is 200±20 MW/cm². The worm-like features illustrated in FIG. 7A reveal the presence of M13 bacteriophages in the control. Nearly all the worm-like features disappear and are replaced by mucus-like structures after laser irradiation as illustrated in FIG. 7B, indicative that the laser irradiation affects the global structure of the viral capsid coat.

FIGS. 7C-7D exemplarily illustrate atomic force microscope images of a tobacco mosaic virus (TMV) sample without laser treatment and a tobacco mosaic virus sample with laser-irradiation by a near-infrared sub-picosecond fiber laser, respectively. TMV is a rod-shape virus whose length can vary depending upon the method of extraction. On average, TMV has a length of about 300 nm, a diameter of about 18 nm and contains a single-stranded RNA. The rectangular white structures correspond to AFM images of TMV in the control as illustrated in FIG. 7C. The narrow worm-like features, which show up only in the laser irradiated sample illustrated in FIG. 7D, represent single-stranded RNAs released from the TMV, presumably as a result of huge vibrations of the TMV's protein shell coherently excited by the laser as discussed below. The laser power density used is 1.0±0.1 GW/cm².

Therefore, AFM images for M13 bacteriophages and TMV clearly demonstrate that near-infrared sub-picosecond fiber laser irradiation can affect the structural integrity of the capsid of a virus. In another embodiment, because the amplitude of the vibrations varies continuously with the laser intensity, as indicated in Equation (1), the method disclosed herein include proper excitation of pathogenic microorganisms, such as use of appropriate laser intensity until the microorganisms reach a state where they are inactivated, but the outer structure of the microorganisms remains intact in an altered or fractured state. It is contemplated that the method can then be used in the manufacture of vaccines.

Laser irradiation experiments have also been carried out on wild-type M13 bacteriophages in addition to the M13 interference-resistant helper phage illustrated in FIG. 6A. The results of these experiments indicate that the threshold laser power intensities for inactivation of M13 bacteriophage and M13 interference-resistant helper phage are the same within the experimental variance. These experimental results suggest that the method disclosed herein can overcome limitations with current therapeutics that arise due to mutations. This is due to the fact that the excited coherent acoustic vibrations induced in the capsids of the M13 phages are usually of a long wavelength; as a result they are relatively insensitive to minor local changes such as those due to mutations.

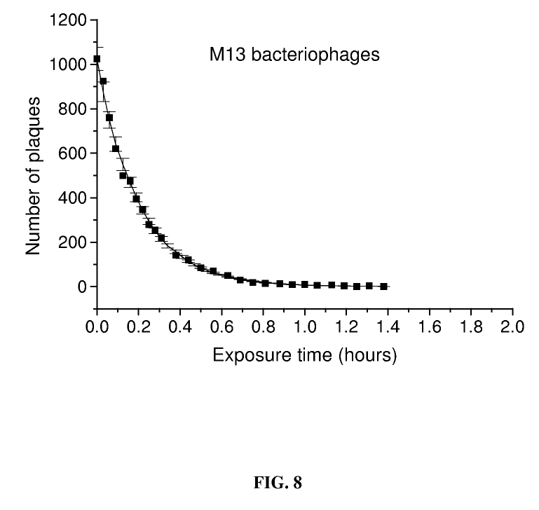

FIG. 8 exemplarily illustrates a graphical representation showing number of plaques for a M13 bacteriophage sample as a function of the exposure time to radiation by a near-infrared sub-picosecond fiber laser. The laser power density used is 100±10 MW/cm². The inactivation is approximately exponential with a time constant of about 0.2 hours. The number of viral particles is reduced to less than about 10% after 0.5 hour of exposure to laser irradiation, and to less than about 0.5% after 1 hour of laser exposure time. The efficiency of inactivation depends on how efficient the viral particle is placed within the effective volume of the near-infrared sub-picosecond fiber laser in the vial. More efficient magnetic stirring gives rise to more efficient inactivation.

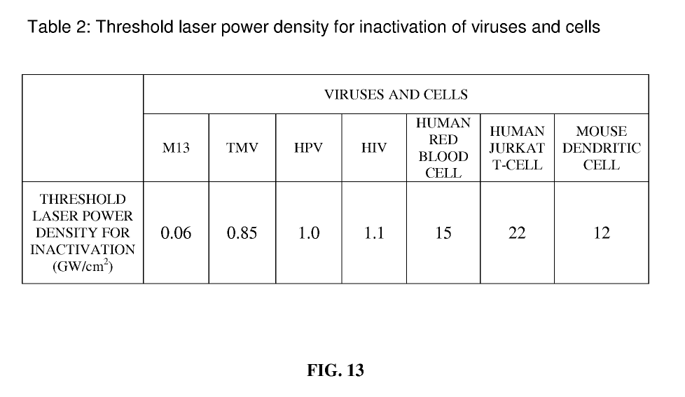

The effects of the near-infrared subpicosecond fiber laser light on other microorganisms besides viruses have also been evaluated. FIG. 13 exemplarily illustrates a table showing threshold laser power density for the inactivation of a variety of viruses and cells by a near-infrared sub-picosecond fiber laser. The table illustrated in FIG. 13 summarizes the threshold laser power density for inactivation of a variety of microorganisms, including human red blood cells, human Jurkat cells and mouse dendritic cells. It has been found that much higher laser power intensities are necessary to inactivate these mammalian cells. These results indicate that there exists a window in laser power density or equivalently, laser intensity (because the same laser is used for the experiments) which is bounded approximately by 1 GW/cm² and 10 GW/cm², that allows the inactivation of unwanted microorganisms such as viruses while leaving useful materials like mammalian cells unharmed.

Therefore the near-infrared sub-picosecond fiber laser, if appropriately manipulated, can be used to selectively kill pathogens with minimal damage to sensitive materials. It is this selectivity of the method disclosed herein that distinguishes our approach from currently available methods. The photonic approach in the method disclosed herein can be used for the disinfection of viral pathogens in blood products and for the treatment of blood-borne viral diseases performed as a dialysis process in the clinic with minimal side effects.

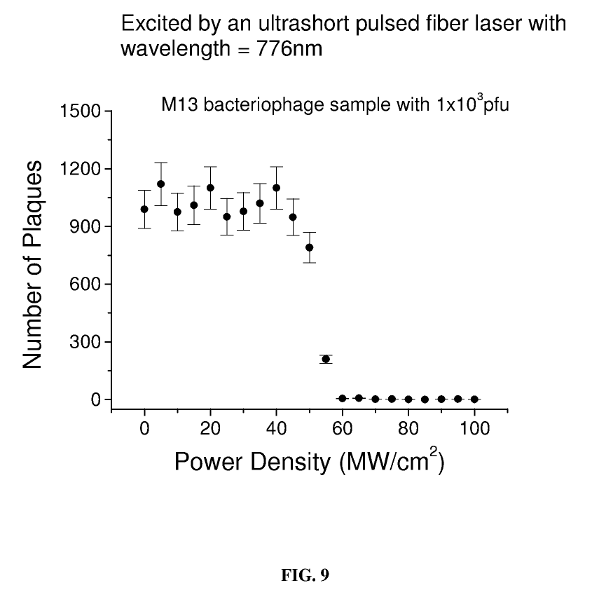

FIG. 9 exemplarily illustrates a graphical representation showing the number of plaques for a M13 bacteriophage sample as a function of the excitation laser power density of a near-infrared subpicosecond fiber laser. FIG. 9 shows the dependence of inactivation of a M13 bacteriophage sample on the power density of the excitation laser. The laser exposure time is kept at 10 hours. When the power density is lower than about 40 MW/cm², no inactivation is observed within an experimental variance; however, as the power density was increased to 60 MW/cm² and higher, inactivation is seen to occur. The abrupt separation of laser power density around 60 MW/cm² for the inactivation of M13 bacteriophage is consistent with the argument that damage on the capsid by ISRS process is the cause of inactivation.

### Example 3

This example demonstrates the inactivation of both E-coli and salmonella bacteria by a visible femtosecond laser. The excitation source employed in this example is the output of the second harmonic generation system (SHG) of a diode-pumped CW mode-locked Ti-sapphire laser. The excitation laser is chosen to operate at a wavelength of 425 nm with an average power of about 50 mW. The excitation laser has a pulse width of full-width at half maximum (FWHM)≅100 fs. An achromatic focus length (f=75 cm) is used to focus the laser beam into the sample area. The relatively uniformed laser-focused volume, which is the active volume for the interaction of the laser with the bacterial samples through ISRS, approximated a cylinder having approximately 100 μm in diameter and 1.5 cm in height. In order to facilitate the interaction of the laser with bacteria which are inside a glass cuvette and diluted in 0.1 ml water, a magnetic stirrer 306 is set up so that the bacteria enter the laser-focused volume as described above and interact with the photons. The laser-irradiated bacteria samples contain about 1×10⁹/ml. The assays are performed on the laser-irradiated samples after proper dilution. The typical exposure time of the sample to laser irradiation is about 1 hour. A thermal couple is used to monitor the temperature of the sample to ensure that the results are not due to heating effects. The increase of the temperature of the bacterial samples is less than 2° C. after 1 hour's laser irradiation. The experimental results are obtained at T=25° C. and with the single laser beam excitation.

After proper dilution, the treated and control samples are spread uniformly over the agar plates. These plates are incubated in an incubator for about 12 hours. The number of bacterial colonies on the plate reflects the number of surviving bacteria.

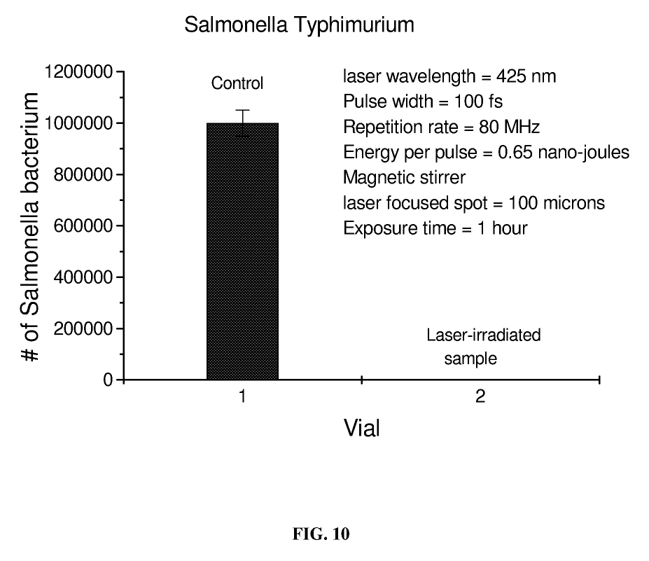

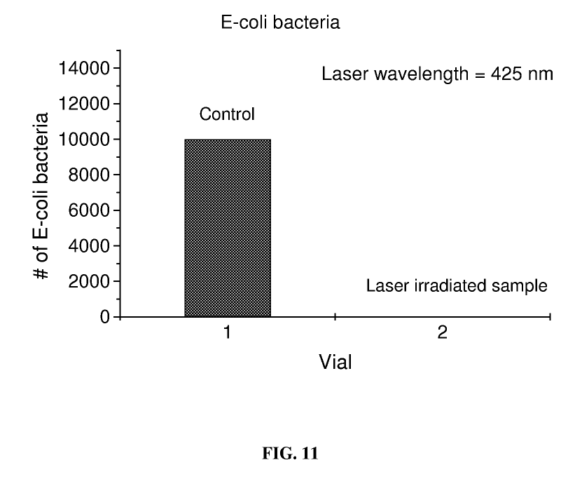

FIG. 10 exemplarily illustrates a graphical representation of two assays showing the number of salmonella bacteria for the control and laser irradiated samples by a visible femtosecond laser as indicated. The bacterial load was found to be reduced by a factor of about 10⁵. FIG. 11 shows the number of bacterial colonies of an E-coli bacterial sample for control (without laser irradiation) and a sample with laser irradiation by a visible femtosecond laser, respectively. The bacterial load was found to be reduced by at least 4 orders of magnitude.

The foregoing examples have been provided merely for the purpose of explanation and in no way are to be construed as limiting of the present invention. While the invention has been described with reference to various embodiments, it is understood that the words, which have been used herein, are words of description and illustration, rather than words of limitation. Additionally, although the invention has been described herein with reference to particular means, materials and embodiments, the invention is not intended to be limited to the particulars disclosed herein; rather, the invention extends to all functionally equivalent structures, methods and uses, such as are within the scope of the appended claims. It will be appreciated by those skilled in the art, having the benefit of the teachings of this specification, that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. A method for selectively inactivating microorganisms while leaving mammalian cells unharmed, comprising:
exciting said microorganisms in a fluid and/or a tissue into vibrational states with a single femtosecond laser beam of radiation at a wavelength in a range of an electromagnetic spectrum where water is substantially transparent, wherein said vibrational states of said excited microorganisms are high amplitude, low-frequency acoustic vibrations on an outer structure of said microorganisms that diminish activity of said microorganisms;
whereby manipulation and control of said femtosecond laser enable selective inactivation of pathogenic microorganisms while leaving said mammalian cells unharmed.

2. The method of claim 1, wherein said fluid is one of water, whole blood, and blood components in their buffer solutions.

3. The method of claim 1, wherein said excitation of said microorganisms results from an impulsive stimulated Raman scattering process.

4. The method of claim 1, wherein said outer structure of said microorganisms is one of a protein shell of a virus and a lipid bi-layer of a bacterium.

5. The method of claim 1, wherein said single femtosecond laser beam is a single laser beam produced by one of a continuous wave mode-locked titanium-sapphire laser, a fiber laser, and an amplifier laser system on which a continuous wave mode-locked laser is based, with pulse width that is less than one picosecond.

6. The method of claim 1, wherein said electromagnetic spectrum where said water is substantially transparent covers a range of electromagnetic waves with wavelength from one of near-infrared to visible spectrum and about 400 nanometers to about 1.3 micrometers.

7. The method of claim 1, wherein said low-frequency acoustic vibrations correspond to vibrational frequency from about 1 gigahertz to about 1000 gigahertz.

8. The method of claim 1, wherein said microorganisms are viruses, bacteria, and protozoa.

9. The method of claim 1, wherein said manipulation and control of said femtosecond laser comprises properly choosing pulse width, wavelength, repetition rate, and power density of said femtosecond laser.

10. The method of claim 1, wherein said generation of said high-amplitude, low-frequency acoustic vibrations on said outer structure of said microorganisms is used in manufacturing a vaccine.
